(19) United States
(12) Patent Application Publication
(10) Pub. No.: US 2008/0299198 A1
(43) Pub. Date: Dec. 4, 2008

(54) Polystyrene sulfonate polymer tablets, their preparation and use

(76) Inventors: Chris Ho, Westborough, MA (US); Jugminder Chawla, Waltham, MA (US); Hitesh R. Bhagat, Wayland, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/899,405

(22) Filed: Sep. 5, 2007

Related U.S. Application Data
(60) Provisional application No. 60/842,441, filed on Sep. 6, 2006.

(51) Int. Cl. A61K 9/28; A61K 47/30; A61K 9/14; A61K 9/20; A61P 31/04

(52) U.S. Cl. 424/474; 514/772.3; 424/501; 424/464

(57) ABSTRACT

The present invention provides tablets containing at least about 70% of polystyrene sulfonate polymer, binder and moisture. The invention further relates to methods of treating medical conditions including antibiotic-associated diarrhea such as that caused by Chlostridium difficile, comprising administration of the tablets to a subject in need thereof. Furthermore, pharmaceutical blends and methods of preparing the tablets are disclosed.

### RELATED APPLICATION

This application claims the benefit of U.S. Provisional Application No. 60/842,441 filed on Sep. 6, 2006.

The entire teachings of the above application are incorporated herein by reference.

### BACKGROUND OF THE INVENTION

Polystyrene sulfonate polymer has been described for the treatment of various medical conditions, including antibiotic-associated diarrhea (AAD). AAD represents a serious medical condition that is caused by pathogenic toxins, such as those released by Clostridium difficile.

There is a need to provide tablets with high loading of polystyrene sulfonate polymer as therapeutic agent, which minimize the number of tablets to be administered to the patient, which can be coated, which are easy to administer orally, and which are stable upon production and storage.

### SUMMARY OF THE INVENTION

The present invention provides tablets of acceptable size with high loading of polystyrene sulfonate polymer that solve the problems of the prior art.

One embodiment of the invention is a tablet comprising at least about 70% by dry tablet weight polystyrene sulfonate polymer, binder and moisture.

In another embodiment of the invention, the tablet comprises about 80% to about 94% by dry tablet weight polystyrene sulfonate polymer, about 6% to about 20% by dry tablet weight hydroxypropyl ether of cellulose characterized by more than 0.4 and not more than 4.6 hydroxypropyl groups per anhydroglucose unit, and about 7% to about 13% by tablet weight moisture.

In another embodiment of the invention, the tablet comprises about 950 mg to about 1070 mg of polystyrene sulfonate polymer, about 118 mg to about 236 mg hydroxypropyl ether of cellulose characterized by more than 0.4 and not more than 4.6 hydroxypropyl groups per anhydroglucose unit, and about 7% to about 13% by tablet weight moisture.

In another embodiment of the invention, the tablet comprises at least about 70% by dry tablet weight polystyrene sulfonate polymer, wherein the hardness of the tablet is about 30 kp to about 70 kp.

Another embodiment of the invention is a pharmaceutical blend comprising at least about 70% by dry blend weight polystyrene sulfonate polymer and a binder.

Another embodiment of the invention is a method of preparing a tablet containing polystyrene sulfonate polymer. The method comprises the step of compressing the pharmaceutical blends disclosed herein with a compression force of about 25 kN to about 60 kN to form a tablet.

Another embodiment of the invention is a method of treating a bacterial infection in a subject, the bacterial infection being characterized by release of a pathogenic toxin, comprising the step of administering to the subject a tablet disclosed herein.

Another embodiment of the invention is a method of treating antibiotic-associated diarrhea in a subject comprising administering to said subject a tablet disclosed herein.

Yet another embodiment of the invention is a method of treating C. difficile associated diarrhea in a subject comprising administering to said subject a tablet disclosed herein.

### DETAILED DESCRIPTION OF THE INVENTION

It has been found that tablets comprising polystyrene sulfonate polymer, binder and moisture can be prepared using conventional pharmaceutical tablet manufacturing equipment to have acceptable sizes with high drug loading.

As used herein the term “acceptable size” refers to tablet dimensions sufficiently small such that the tablet is easily swallowable by a subject.

As used herein a “subject” is a mammal, most preferably a human, but can also be an animal in need of veterinary treatment, such as a companion animal (e.g., dogs, cats, and the like), a farm animal (e.g., cows, sheep, pigs, horses, and the like) or a laboratory animal (e.g., rats, mice, guinea pigs, and the like).

As used herein the term “drug loading” refers to the percentage of drug, for example, polystyrene sulfonate polymer in the tablet.

“Tablet weight” as used herein, is the weight of a tablet without a coating.

“Dry tablet weight” as used herein, is the tablet weight without moisture. The moisture content in a tablet, pharmaceutical blend or tablet ingredient can be obtained by determining the Loss On Drying (LOD) using methods known in the art. For example, a tablet that contains 10% by dry tablet weight of a compound, wherein the dry tablet weight is 1 g, contains 100 mg of the compound. Furthermore, if the tablet weight is 1.25 g, the tablet contains 250 mg moisture, that is, 20% by tablet weight moisture.

As used herein, “pharmaceutical blend” (hereinafter also “blend”) refers to a powder mixture of one or more solid pharmaceutically active agents and one or more solid pharmaceutically suitable inactive agents (e.g., excipients, binders, etc.). Specifically, a pharmaceutical blend is a mixture of solid compounds of small particle sizes, that is, a powder. Typically, a pharmaceutical blend or a portion of a pharmaceutical blend can be compressed to form a tablet. Pharmaceutical blends can be characterized by their compactibility. As used herein, “compactibility” is the ratio of tablet hardness to the compression force applied to the pharmaceutical blend to form a tablet. Preferably, pharmaceutical blends of the present invention may have a compactibility of about 9 N/kN to about 20 N/kN.

“Blend weight” as used herein, is the weight of a pharmaceutical blend.

“Dry blend weight” as used herein, is the blend weight without moisture.

“Moisture” as used herein, represents water that can optionally have chemical or physical interactions with tablet or pharmaceutical blend ingredients. The compactibility of a pharmaceutical blend is dependent in part on the amount of moisture present. As such, the amount of moisture in a pharmaceutical blend can be adjusted such that the pharmaceutical blend can be readily compressed into tablets.

Typically, tablets may comprise about 7% to about 13% by tablet weight moisture. More specifically, tablets may comprise about 8% to about 12% by tablet weight moisture. Most specifically, tablets may comprise about 9% by tablet weight moisture.

Pharmaceutical blends of the present invention optionally comprise moisture. Specifically, pharmaceutical blends typically comprise about 7% to about 13% by tablet weight moisture. More specifically, pharmaceutical blends may comprise about 8% to about 12% by tablet weight moisture. Most specifically, pharmaceutical blends may comprise about 9% by tablet weight moisture.

A “binder” as used herein is a tablet or pharmaceutical blend ingredient that takes up space in a tablet or pharmaceutical blend and holds a tablet together after a corresponding pharmaceutical blend has been compressed. Suitable binders allow tablets to comprise at least about 70% of polystyrene sulfonate polymer. Two commonly used binders are hydroxypropyl ether of cellulose characterized by more than 0.4 and not more than 4.6 hydroxypropyl groups per anhydroglucose unit (hereinafter, “hydroxypropyl ether of cellulose”) and polyethylene glycol. More specifically, tablets or pharmaceutical blends may comprise about 5% to about 30% by dry tablet weight hydroxypropyl ether of cellulose or polyethylene glycol. Even more specifically, tablets or pharmaceutical blends may comprise about 5% to about 20%, about 5.2% to about 20%, about 5.4% to about 20%, about 5.6% to about 20%, about 5.8% to about 20%, about 6% to about 20%, about 6.2% to about 20%, about 6.4% to about 20%, or about 6.5% to about 20% by dry tablet weight hydroxypropyl ether of cellulose or polyethylene glycol. Most specifically, tablets or pharmaceutical blends may comprise about 6.6% by dry tablet weight hydroxypropyl ether of cellulose or polyethylene glycol.

Binder can be characterized by its particle size distribution. “Particle size distribution” of the binder as used herein, refers to the distribution of particle sizes, wherein each particle comprises one or more molecules of the binder compound. Several methods for measuring particle sizes and determining mean particle sizes are known in the art. “Mean particle size” is determined by aerosizing as known in the art, and “Volume weighted mean particle size” refers to the measurement and determination based on a sieve analysis as known in the art. Preferably, the volume weighted mean particle size of the binder may be from about 10 μm to about 100 μm. More preferably, the volume weighted mean particle size may be from about 20 μm to about 50 μm. More preferably, the volume weighted mean particle size may be from about 35 μm to about 40 μm.

Tablets and pharmaceutical blends of the present invention comprise polystyrene sulfonate polymer. Specifically, tablets or pharmaceutical blends may comprise at least 70% by dry tablet weight polystyrene sulfonate polymer. More specifically, tablets or pharmaceutical blends may comprise about 70% to about 94% by dry tablet weight polystyrene sulfonate polymer. Even more specifically, tablets or pharmaceutical blends may comprise about 70% to about 93.5%, about 75% to about 93.5%, about 80% to about 93%, or about 85% to about 90% by dry tablet weight polystyrene sulfonate polymer. Most specifically, tablets or pharmaceutical blends may comprise about 92.8% by dry tablet weight polystyrene sulfonate polymer.

Preferably, tablets may comprise between about 600 mg and about 1200 mg, about 700 mg and about 1200 mg, about 800 mg and about 1100 mg, about 950 mg and about 1070 mg polystyrene sulfonate polymer. Most preferably, tablets may comprise about 1000 mg polystyrene sulfonate polymer.

“Polystyrene sulfonate polymer” as used herein, includes polystyrene sulfonic acid (i.e., a polymeric acid) and pharmaceutically acceptable salts thereof. “Physiologically acceptable” means suitable for pharmaceutical use. The term “salt” refers to the partially or fully deprotonated form of the polymeric acid in combination with a pharmaceutically acceptable cation. Suitable cations include but are not limited to alkali metal ions, such as sodium, potassium and cesium ions, alkaline earth ions, such as calcium and magnesium ions, transition metal ions and unsubstituted and substituted (primary, secondary, tertiary and quaternary) ammonium ions. In one embodiment, the cation is a polyvalent metal ion, such as Ca²⁺, Mg²⁺, Zn²⁺, Al³⁺, Bi³⁺, Fe²⁺ or Fe³⁺. Typically, polystyrene sulfonate polymer can be sodium polystyrene sulfonate, potassium polystyrene sulfonate, a co-polymer of sodium polystyrene sulfonate and potassium polystyrene sulfonate, a mixture of sodium polystyrene sulfonate and potassium polystyrene sulfonate, or a mixture of co-polymers of sodium polystyrene sulfonate and potassium polystyrene sulfonate.

Polystyrene sulfonate polymers of the present invention can be prepared by the methods previously described. For example, U.S. Pat. Nos. 6,270,755 and 6,290,946, and the International Publication WO 2004/009100A1 describe methods of synthesis of polystyrene sulfonate polymers by polymerizing styrene sulfonate (e.g., Examples 8 and 12 of U.S. Pat. No. 6,290,946). The entire teachings of the above documents are incorporated herein by reference. Preferably, polystyrene sulfonate polymer can be sodium polystyrene sulfonate, that is, a polymer consisting of repeat units of the following Structural Formula (I):

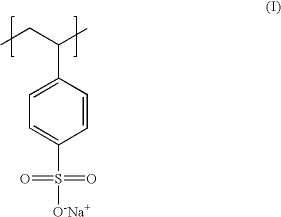

Alternatively, polystyrene sulfonate polymer can be potassium polystyrene sulfonate, that is, a polymer consisting of repeat units of the following Structural Formula (II):

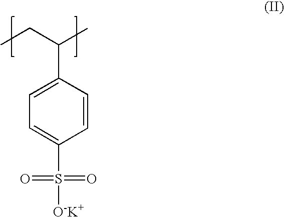

In another alternative, polystyrene sulfonate polymer can be a co-polymer of sodium polystyrene sulfonate and potassium polystyrene sulfonate such as TOLEVAMER, that is, a polystyrene sulfonate copolymer. Polystyrene sulfonate copolymers may comprise or may consist of repeat units represented by Structural Formula (I) and Structural Formula (II). Preferably, about 20% to about 70% of the repeat units may be represented by Structural Formula (II) and about 30% to about 80% of the repeat units may be represented by Structural Formula (I). Alternatively, about 30% to about 45% of the repeat units may be represented by Structural Formula (II) and about 55% to about 70% of the repeat units may be represented by Structural Formula (I), about 35% to about 40% of the repeat units may be represented by Structural Formula (II) and about 60% to about 65% of the repeat units may be represented by Structural Formula (I), or about 37% of the repeat units may be represented by Structural Formula (II) and about 63% of the repeat units may be represented by Structural Formula (I).

In yet another alternative, polystyrene sulfonate polymer can be a mixture of sodium polystyrene sulfonate and potassium polystyrene sulfonate. Polystyrene sulfonate mixtures may comprise about 20% to about 70%, about 30% to about 45%, about 35% to about 40%, or about 37% potassium polystyrene sulfonate and about 30% to about 80%, about 55% to about 70%, about 60% to about 65%, or about 63% of sodium polystyrene sulfonate.

The weight of the polystyrene sulfonate polymer may typically be greater than 100,000 Daltons and preferably greater than 400,000 Daltons, such that the polymer is large enough not to be absorbed by the gastrointestinal tract. The upper limit of the weight is not crucial. Typically, polystyrene sulfonate polymers of the present invention may weigh from about 100,000 Daltons to about 5,000,000 Daltons, or about 200,000 Daltons to about 2,000,000 Daltons, or about 300,000 Daltons to about 1,500,000 Daltons. The polystyrene sulfonate polymers can either be crosslinked or uncrosslinked, but are preferably uncrosslinked and water soluble.

Polystyrene sulfonate polymer can be characterized by its particle size distribution. “Particle size distribution” of polystyrene sulfonate polymer as used herein, refers to the distribution of particle sizes, wherein each particle comprises one or more polystyrene sulfonate polymer molecules. Several methods for measuring particle sizes and determining mean particle sizes are known in the art. “Mean particle size” is determined by aerosizing as known in the art, and “Volume weighted mean particle size” refers to the measurement and determination based on a sieve analysis as known in the art. Preferably, the mean particle size may be from about 15 μm to about 70 μm and the volume weighted mean particle size may be from about 30 μm to about 90 μm. More preferably, the mean particle size may be from about 25 μm to about 50 μm and the volume weighted mean particle size may be from about 40 μm to about 60 μm.

Tablets and pharmaceutical blends of the present invention may further comprise one or more pharmaceutically acceptable excipients. “Excipients” as used herein include, but are not limited to, fillers or diluents, disintegrants, glidants, lubricants, anti-adherents, flavours and colourants (see also “Handbook of Pharmaceutical Excipients”, 5th edition edited by Raymond C. Rowe and others. London; Greyslake, Ill.: Pharmaceutical Press; Washington, D.C.: American Pharmacists Association, 2006)

A “glidant” is a compound that can be added to a pharmaceutical blend to improve the flowability of the pharmaceutical blend used to form a tablet. Examples of glidants are calcium phosphate (tribasic), calcium silicate, cellulose (powdered), microcrystalline cellulose (e.g., Emcocel® SP15), magnesium silicate, magnesium trisilicate, silicon dioxide, colloidal silicon dioxide (e.g., Cab-O-Sil®), starch (e.g., Starch-1500, i.e., pre-gelatinized starch), and talc. Preferably, the tablets may comprise colloidal silicon dioxide as glidant. Specifically, the tablets may comprise about 0% to about 1% by dry tablet weight of a glidant such as colloidal silicon dioxide. More specifically, the tablets may comprise about 0.02% to about 1% by dry tablet weight of a glidant such as colloidal silicon dioxide. Even more specifically, the tablets may comprise about 0.02% to about 0.5% by dry tablet weight of a glidant such as colloidal silicon dioxide. Yet even more specifically, the tablets may comprise about 0.02% to about 0.2% by dry tablet weight or about 0.05% to about 0.15% by dry tablet weight of a glidant such as colloidal silicon dioxide. Most specifically, the tablets may comprise about 0.1% by dry tablet weight of a glidant such as colloidal silicon dioxide.

A “lubricant” is a compound which can be added to a powder blend to prevent the compacted pharmaceutical blend from sticking to the equipment during the tabletting process. It also aids the ejection of the tablet from the dies, and in some cases may help improve flow of the pharmaceutical blend. Examples of lubricants are calcium stearate, D-(+)-glucose monohydrate, glyceryl monostearate, glyceryl behenate, glyceryl palmitostearate, hydrogenated castor oil, hydrogenated vegetable oil (type I), light mineral oil, magnesium lauryl sulfate, magnesium stearate, mannitol, medium-chain triglycerides, mineral oil, poloxamer, polyvinyl alcohol, potassium chloride, sodium benzoate, sodium chloride, sodium lauryl sulphate, sodium stearyl fumarate (e.g., Pruv®), talc, starch (e.g., Starch-1500, i.e., pre-gelatinized starch), stearic acid, zinc and stearate. Preferably, the tablets may comprise sodium stearyl fumarate as lubricant. Specifically, the tablets may comprise about 0% to about 5% by dry tablet weight of a lubricant such as sodium stearyl fumarate. More specifically, the tablets may comprise about 0.1% to about 5% or about 0.1% to about 2.5% by dry tablet weight of a lubricant such as sodium stearyl fumarate. Even more specifically, the tablets may comprise about 0.1% to about 1% or about 0.25% to about 0.75% by dry tablet weight of a lubricant such as sodium stearyl fumarate. Most specifically, the tablets may comprise about 0.5% by dry tablet weight of a lubricant such as sodium stearyl fumarate.

Tablets of the present invention may further comprise one or more additional drugs, such as antibiotics, anti-inflammatory agents or analgesics.

Tablets of the present invention may further comprise a coating. A “coating” is a material that surrounds the compressed tablet ingredients. Suitable coatings are stable and strong enough to survive the handling of the tablet, prevent tablets from sticking together during the coating process, provide a smooth tablet surface that makes large tablets easier to swallow and do not substantially limit the dissolution of the tablet. Examples of coating formulations that may be used to coat tablets are LustreClear®, Eudragit® EPO, hydroxypropylmethylcellulose (HPMC) based coatings, Kollicoat® IR and Opadry®-II. Suitable coating systems carry high solids content and require low drying temperatures. Preferably, the coating formulation may be Opadry®-II.

The tablets of the present invention can be characterized by their hardness. “Hardness” as used herein, is a measure of the force (measured herein in units of “kp”, that is, kilopond corresponding to about 9.8 Newton) needed to fracture a tablet when such tablet is placed lengthwise on a Hardness Tester such as those known in the art. Preferably, tablets may have a hardness from about 30 kp to about 70 kp. More preferably, tablets may have a hardness from about 35 kp to about 68 kp. Most preferably, the hardness may be from about 40 kp to about 66 kp.

In a specific embodiment of the invention, the tablet comprises at least about 70% by dry tablet weight polystyrene sulfonate polymer, about 5% to about 30% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet may be about 9% by tablet weight.

In another specific embodiment, the tablet comprises about 70% to about 94% by dry tablet weight polystyrene sulfonate polymer, about 5% to about 30% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 70% to about 93.5% by dry tablet weight polystyrene sulfonate polymer, about 5.5% to about 30% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 75% to about 93.5% by dry tablet weight polystyrene sulfonate polymer, about 5.5% to about 25% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 80% to about 94% by dry tablet weight polystyrene sulfonate polymer, about 5% to about 20% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 80% to about 93.5% by dry tablet weight polystyrene sulfonate polymer, about 5.5% to about 20% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 80% to about 93.5% by dry tablet weight polystyrene sulfonate polymer, about 6.5% to about 20% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 80% to about 93% by dry tablet weight polystyrene sulfonate polymer, about 6% to about 20% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 85% to about 90% by dry tablet weight polystyrene sulfonate polymer, about 9% to about 20% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

In another specific embodiment, the tablet comprises about 92.8% by dry tablet weight polystyrene sulfonate polymer, about 6.6% by dry tablet weight hydroxypropyl ether of cellulose or polyethylene glycol, and moisture. The moisture content of the tablet is typically about 7% to about 13% by tablet weight, more typically, the moisture content of the tablet is about 8% to about 12% by tablet weight, even more typically, the moisture content of the tablet is about 9% by tablet weight.

Another embodiment of the present invention is a tablet comprising about 80% to about 94% by dry tablet weight polystyrene sulfonate polymer, about 5% to about 20% by dry tablet weight hydroxypropyl ether of cellulose, and about 7% to about 13% by tablet weight moisture.

In a more specific embodiment, the tablet comprises about 92.8% by dry tablet weight polystyrene sulfonate polymer, about 6.6% by dry tablet weight hydroxypropyl ether of cellulose, about 0.1% by dry tablet weight colloidal silicon dioxide, about 0.5% by dry tablet weight sodium stearyl fumarate, and about 9% by tablet weight moisture.

In another embodiment of the present invention, the tablet comprises about 950 mg to about 1070 mg of polystyrene sulfonate polymer, about 118 mg to about 236 mg hydroxypropyl ether of cellulose, and about 7% to about 13% by tablet weight moisture.

In a more specific embodiment, the tablet comprises about 1000 mg of polystyrene sulfonate polymer, about 180.18 mg hydroxypropyl ether of cellulose, about 1.287 mg colloidal silicon dioxide, about 6.435 mg sodium stearyl fumarate, and about 9% by tablet weight moisture.

In another embodiment, the tablet of the present invention is as described in any one of the preceding fourteen paragraphs, further characterized by a hardness that, preferably, is about 30 kp to about 70 kP, more preferably, is about 35 kp to about 68 kp and, most preferably, is about 40 kp to about 66 kp.

Other embodiments of the invention are directed to pharmaceutical blends comprising polystyrene sulfonate polymer. In one embodiment, the pharmaceutical blends comprise the same ingredients as the tablets described in any one of the preceding fifteen paragraphs, wherein polystyrene sulfonate polymer in the blends is further characterized by having, preferably, a mean particle size from about 15 μm to about 70 μm and a volume weighted mean particle size from about 30 μm to about 90 μm and, more preferably, a mean particle size from about 25 μm to about 50 μm and a volume weighted mean particle size from about 40 μm to about 60 μm.

In related embodiments, the pharmaceutical blends comprise the same ingredients as the tablets described in any one of the preceding ten paragraphs, wherein polystyrene sulfonate polymer in the blends is further characterized by having, preferably, a mean particle size from about 15 μm to about 70 μm and a volume weighted mean particle size from about 30 μm to about 90 μm and, more preferably, a mean particle size from about 25 μm to about 50 μm and a volume weighted mean particle size from about 40 μm to about 60 μm, and hydroxypropyl ether of cellulose is further characterized by having, preferably, a particle size distribution with a volume weighted mean particle size of about 20 μm to about 100 μm and, more preferably, a volume weighted mean particle size of about 35 μm to about 40 μm.

In a specific embodiment, the pharmaceutical blend comprises at least about 70% by dry blend weight polystyrene sulfonate polymer and about 5% to about 30% by dry blend weight hydroxypropyl ether of cellulose or polyethylene glycol.

In another specific embodiment, the pharmaceutical blend comprises about 80% to about 94% by dry blend weight polystyrene sulfonate polymer and about 5% to about 20% by dry blend weight hydroxypropyl ether of cellulose or polyethylene glycol.

In another specific embodiment, the pharmaceutical blend comprises about 92.8% by dry blend weight polystyrene sulfonate polymer and 6.6% by dry blend weight hydroxypropyl ether of cellulose or polyethylene glycol.

Further embodiments of the present invention are directed to a method of preparing a tablet containing polystyrene sulfonate polymer. The methods presented herein comprise the step of compressing a pharmaceutical blend of the present invention with a compression force suitable to form a tablet. Preferably, the compression force is from about 25 kN to about 60 kN. More preferably, the compression force is about 35 kN to about 50 kN.

The methods for preparing a tablet containing polystyrene sulfonate polymer may further comprise pharmaceutical blends that are pre-compressed.

As used herein “precompression force” refers to the force that is used to place the pharmaceutical blend into the die and de-airate it. Preferably, pharmaceutical blends may be pre-compressed with a precompression force of about 5 kN to about 30 kN. More preferably, the pharmaceutical blends may be pre-compressed with a pre-compression force of about 10 kN to about 20 kN. Most preferably, pharmaceutical blends may be pre-compressed with a precompression force of about 15 kN.

The pharmaceutical blends of the present invention can be compressed into tablets using conventional manufacturing equipment. Suitable press toolings form tablets of acceptable shape and form. Preferably, an oval, shallow B-press tooling may be used, resulting in oval tablets.

Further embodiments of the present invention are directed to methods of treating a medical condition in a subject, comprising administering to the subject a tablet of the present invention. “Medical conditions” as used herein, can be, but are not limited to, bacterial infections, antibiotic associated diarrheas (AADs), or inflammatory colitis. Preferably, the bacterial infections are characterized by release of a pathogenic toxin. One example of an antibiotic associated diarrhea is Clostridium difficile associated diarrhea (CDAD). As used herein, the term “treating a medical condition” refers to inhibiting the activity of a pathogenic toxin which is associated with the development of a particular medical condition and may include: prophylactic treatment of those subjects susceptible to the medical condition; treatment at the initial onset of the medical condition; treatment of an ongoing medical condition; and treatment of a relapsing medical condition in susceptible subjects. As used herein a “susceptible” subject is a subject capable of developing a medical condition or having a relapse of a medical condition for any reason including use of broad spectrum antibiotics which may disrupt the normal flora leading, for example, to CDAD, or exposure to bacteria causing such a medical condition. The pathogenic toxin can be inhibited by any mechanism, including, but not limited to, binding of the pathogenic toxin by polystyrene sulfonate polymer administered to a subject in the form of a tablet of the present invention.

“Pathogenic toxin” as used herein is an endotoxin or exotoxin released by a microorganism, such as a bacterium, a fungus, a protozoan or a virus, preferably, the pathogenic toxin may be released by a bacterium. Pathogenic toxins include, but are not limited to, toxins produced by Streptococcus spp., including Streptococcus pneumoniae, Streptococcus pyogenes and Streptococcus Sanguis; Salmonella spp., including Salmonella enteritidis; Campylobacter spp., including Campylobacter jejuni; Escherichia spp., including E. coli; Clostridia spp., including Clostridium difficile and Clostridium botulinum; Staphylococcus spp., including Staphylococcus aureus; Shigella spp., including Shigella dysenteriae; Pseudomonas spp., including Pseudomonas aeruginosa; Bordatella spp., including Bordatella pertussis; Listeria spp., including Listeria monocytogenes; Vibrio cholerae; Yersinia spp., including Yersinia enterocolitica; Legionella spp., including Legionella pneumophilia; Bacillus spp., including Bacillus anthracis; Helicobacter spp.; Corynebacteria spp.; Actinobacillus spp.; Aeromonas spp.; Bacteroides spp. including Bacteroides fragilis; Neisseria spp, including N. meningitidis; Moraxella spp., such as Moravella catarrhalis and Pasteurella spp. Also included are protozoal toxins, such as toxins produced by Entameoba histolytica and Acanthameoba; and parasitic toxins. Of particular pathogenic importance are Escherichia coli, for example, E. coli strains 06:H-, 0157:H7, 0143 and other clinical isolates, and Clostridium difficile. Enterohemorrhagic E. coli (EHEC), such as 0157:H7, can cause a characteristic nonfebrile bloody diarrhea known as hemorrhagic colitis. EHEC produce high levels of one or both of two related cytotoxins which resemble a Shiga toxin in structure and function and are referred to as Shiga-like toxins (SLT I or SLT II). These Shiga-like toxins are believed to damage the intestinal mucosa, resulting in the manifestation of hemorrhagic colitis.

In a specific embodiment, the pathogenic toxin is released from Clostridium difficile. C. difficile produces two toxins, Toxin A and Toxin B. Toxin A is an enterotoxin which stimulates infiltration of neutrophils and release of mediators of inflammation, resulting in fluid secretion, altered membrane permeability and hemorrhagic necrosis. Toxin B is a cytotoxin. C. difficile is associated with many cases of antibiotic-associated diarrhea and most cases of pseudomembranous colitis, a severe, potentially fatal inflammation of the colon. Treatment of C. difficile infection typically involves administration of vancomycin or metronidazole.

As used herein “treatment” of C. difficile associated diarrhea (CDAD) includes: prophylactic treatment of subjects susceptible to CDAD; treatment at initial onset of CDAD; treatment of ongoing CDAD and treatment of relapsing CDAD in susceptible patients.

As used herein, a “therapeutically effective amount” is an amount sufficient to inhibit or prevent, partially or totally, tissue damage or other symptoms associated with the action of the toxin within or on the body of the patient or to prevent or reduce the further progression of such symptoms. The amount of pharmaceutically active ingredients (e.g., polystyrene sulfonate polymer) to be administered to a subject in need thereof will be determined on an individual basis and will be determined, at least in part, by consideration of the individual's size, the identity of the known or suspected pathogenic organism (e.g. Chlostridium difficile), the severity of symptoms to be treated and the result sought. The “therapeutically effective number” of tablets to be administered to a subject will be based on the therapeutically effective amount determined as described above.

Polystyrene sulfonate polymers may be administered at a dosage of about 0.1 g/day to about 10 g/day and more preferably from about 1.0 g/day to about 7.0 g/day and even more preferably from about 2.0 g/day to about 6.6 g/day. Most preferably, polystyrene sulfonate polymers may be administered at a dosage of about 3.0 g/day to about 6.0 g/day. For example, for tablets of the present invention with a dosage of about 1 g polystyrene sulfonate polymer, accordingly, about one tenth to about 10 tablets may be administered daily and more preferably about 1 to about 7 tablets may be administered daily and even more preferably about 2 to about 6.6 tablets may be administered daily and most preferably about 3 to about 7 tablets may be administered daily.

The therapeutically effective amount and the corresponding number of tablets can be administered in a single dose or in a series of doses separated by appropriate time intervals, such as hours.

The tablets of the present invention can also be administered in combination with one or more antimicrobial agents, for example, selected from among antibiotics which are known in the art. The antibiotic to be administered is, generally, selected based on the identity or suspected identity of the pathogenic microorganism, as is known in the art. For example, if the pathogenic microorganism is C. parvum, one suitable antibiotic which can be administered in combination with the tablet is paromomycin. The tablet and the antimicrobial agent can be administered simultaneously, for example, in separate dosage forms or in a single dosage form, or in sequence separated by appropriate time intervals.

In another embodiment, the condition to be treated is C. difficile induced gastroenteritis, such as antibiotic-associated diarrhea or pseudomembranous colitis. In this embodiment, the tablets of the present invention can optionally be administered in combination with one or more antibiotic agents which are effective, at least partially, against C. difficile, such as vancomycin and metronidazole.

The term “antimicrobial agent” is intended to include antibacterial agents, antifungal agents, antiseptics and the like. Suitable antimicrobial agents are known in the art and include isoniazid, rifampin, pyrazinamide, ethambutol, erythromycin, vancomycin, tetracycline, chloramphenicol, sulfonamides, gentamicin, amoxicillin, penicillin, streptomycin, p-aminosalicyclic acid, clarithromycin, clofazimine, minocycline, sulfonamides, ethionamide, cycloserine, kanamycin, amikacin, capreomycin, viomycin, thiacetazone, rifabutin and the quinolones, such as ciprofloxacin, ofloxacin and sparfloxicin. The term “antibacterial agent” includes but is not limited to: naturally occurring antibiotics produced by microorganisms to suppress the growth of other microorgansims, and agents synthesized or modified in the laboratory which have either bactericidal or baceriostatic activity, e.g., β-lactam antibacterial agents including, e.g. carbencillim; ampicillin, cloxacillin, oxacillin and pieracillin, cephalosporins and other cephems including, e.g. cefaclor, cefamandole, cefazolin, cefoperazone, ceftaxime, cefoxitin, ceftazidime, ceftriazone and carbapenems including, e.g., imipenem and meropenem; and glycopeptides, macrolides, quinolones (e.g. nalidixic acid), tetracyclines, aminoglycosides (e.g. Gentamicin and Paromomycin) and further includes antifungal agents. In general if an antibacterial agent is bacteriostatic, it means that the agent essentially stops bacterial cell growth (but does not kill the bacteria); if the agent is bacteriocidal, it means that the agent kills bacterial cells (and may stop growth before killing the bacteria).

The invention is described by the following examples which are not intended to be limiting in any way.

### EXAMPLES

### Example 1

The pharmaceutical blends contained 80% by blend weight of sodium polystyrene sulfonate (GT160-246), 16% by blend weight of the respective binder, 3.9% by blend weight of Emcocel® SP15 (microcrystalline cellulose), and 0.1% by blend weight of Pruv® (sodium stearyl fumarate). The blend ingredients were not dried before mixing. Accordingly the pharmaceutical blends contained moisture of about 5% to 6% by blend weight. The pharmaceutical blends were compressed into capsule shaped tablets with a tablet hardness of approximately 30 kp to 50 kp and a tablet weights of about 950 mg. Due to the poor flow properties of most of the blends, the use of a force feeder in the tablet press was advantageous. Three different compression forces (about 30 kN, 37 kN and 45 kN) and three pre-compression forces (about 6 kN, 10 kN and 15 kN) were employed. Physical characteristics of the tablets such as tablet capping and cracking were observed. Binders used in the pharmaceutical blends were HPC LH-22 (low substituted hydroxypropyl ether of cellulose; source: ShinEtsu Chemical Co. Ltd.), HPC LH-32 (source: ShinEtsu Chemical Co. Ltd.), Kollidon® VA-64 (Copovidone; source: BASF), Plasdone® S-630 (source: ISP Technologies Inc.), Methocel® A15 Prem LV (methyl cellulose; source: Dow Chemical Company), PEG-8000 (polyethylene glycol; source: Union Carbide Corporation), and Klucel® EXAF Pharm (hydroxypropyl ether of cellulose; source: Hercules Inc., Aqualon division).

To evaluate the effect of binder particle size, different particle sizes of L-HPC were evaluated (LH-22 and LH-32). The physical characteristics that were determined for tablets (without coating) containing the different binders are shown below (*: actual values varied in the range of ±2 kN; ** for 5 measurements; *** corresponds to one or more cracks occurring on the sides of the tablets):

| | | Approx.* | | | |
|---|---|---|---|---|---|
| | Approx.* | Average | | | |
| | Average | Pre- | Average | | |
| Formulation | Compression | Compression | Ejection | Average** | Crack***/No |
| (Dry Binder) | Force (kN) | Force (kN) | Force (N) | Hardness (kP) | Crack (Y/N) |
| HPC LH-22 | 30.0 | 6.0 | 157.6 | 44.46 ± 2.23 | Y |
| | | 10.0 | 170.3 | 42.14 ± 1.74 | Y |
| | | 15.0 | 171.0 | 38.92 ± 5.70 | Y |
| | 37.0 | 6.0 | 135.7 | 52.16 ± 4.29 | Y |
| | | 10.0 | 122.6 | 51.74 ± 2.41 | Y |
| | | 15.0 | 106.8 | 50.62 ± 4.78 | Y |
| | 45.0 | 6.0 | 86.9 | 59.60 ± 1.73 | Y |
| | | 10.0 | 96.0 | 56.72 ± 3.96 | Y |
| | | 15.0 | 117.4 | 53.60 ± 2.09 | Y |
| HPC LH-32 | 30.0 | 6.0 | 117.0 | 46.48 ± 4.01 | Y |
| | | 10.0 | 124.4 | 43.06 ± 4.21 | Y |
| | | 15.0 | 158.2 | 38.24 ± 2.01 | Y |
| | 37.0 | 6.0 | 136.4 | 54.06 ± 6.77 | Y |
| | | 10.0 | 127.6 | 56.44 ± 4.46 | Y |
| | | 15.0 | 116.1 | 54.44 ± 3.52 | Y |
| | 45.0 | 6.0 | 92.4 | 60.36 ± 4.88 | Y |
| | | 10.0 | 103.0 | 60.60 ± 2.03 | Y |
| | | 15.0 | 109.0 | 60.22 ± 3.09 | Y |
| Klucel ® | 30.0 | 6.0 | 172.7 | 45.60 ± 0.89 | Y |
| EXAF | | 10.0 | 199.7 | 44.70 ± 1.33 | Y |
| | | 15.0 | 207.3 | 43.70 ± 1.39 | N |
| | 37.0 | 6.0 | 191.8 | 47.64 ± 3.88 | N |
| | | 10.0 | 169.8 | 42.14 ± 4.46 | N |
| | | 15.0 | 155.7 | 44.34 ± 1.72 | N |
| | 45.0 | 6.0 | 127.3 | 54.04 ± 1.95 | N |
| | | 10.0 | 127.9 | 55.04 ± 1.41 | N |
| | | 15.0 | 141.0 | 56.48 ± 1.34 | N |
| Kollidon ® | 30.0 | 6.0 | 191.4 | 36.88 ± 2.33 | Y |
| VA-64 | | 10.0 | 189.7 | 36.76 ± 2.92 | Y |
| | | 15.0 | 177.1 | 37.66 ± 2.30 | Y |
| | 37.0 | 6.0 | 144.5 | 44.04 ± 1.77 | Y |
| | | 10.0 | 143.4 | 45.76 ± 1.28 | N |
| | | 15.0 | 140.6 | 46.56 ± 2.11 | Y |
| | 45.0 | 6.0 | 189.1 | 26.40 ± 1.79 | N |
| | | 10.0 | 199.2 | 32.96 ± 1.05 | Y |
| | | 15.0 | 196.4 | 32.96 ± 3.24 | Y |
| Methocel ® | 30.0 | 6.0 | 135.7 | 39.92 ± 2.21 | Y |
| A15 Prem LV | | 10.0 | 141.6 | 44.06 ± 1.16 | Y |
| (Methyl | | 15.0 | 130.7 | 41.74 ± 5.74 | Y |
| cellulose) | 37.0 | 6.0 | 107.4 | 52.28 ± 4.10 | Y |
| | | 10.0 | 107.2 | 55.12 ± 2.38 | Y |
| | | 15.0 | 92.5 | 56.36 ± 3.10 | Y |
| | 45.0 | 6.0 | 66.2 | 61.88 ± 2.54 | Y |
| | | 10.0 | 71.8 | 57.16 ± 2.32 | Y |
| | | 15.0 | 82.0 | 59.28 ± 4.38 | Y |
| Plasdone ® | 30.0 | 6.0 | 95.2 | 39.86 ± 3.84 | Y |
| S-630 | | 10.0 | 108.4 | 37.84 ± 3.45 | Y |
| | | 15.0 | 111.6 | 36.90 ± 3.03 | Y |
| | 37.0 | 6.0 | 86.2 | 41.92 ± 5.00 | Y |
| | | 10.0 | 38.6 | 46.74 ± 1.59 | Y |
| | | 15.0 | 67.7 | 39.60 ± 3.87 | Y |
| | 45.0 | 6.0 | 53.0 | 47.72 ± 3.03 | Y |
| | | 10.0 | 54.7 | 49.28 ± 2.09 | Y |
| | | 15.0 | 59.2 | 45.64 ± 2.58 | Y |
| PEG 8000 | 30.0 | 6.0 | 128.7 | 31.92 ± 2.42 | Y |
| | | 10.0 | 128.2 | 35.40 ± 2.81 | Y |
| | | 15.0 | 136.3 | 31.84 ± 3.23 | N |
| | 37.0 | 6.0 | 115.0 | 39.46 ± 2.45 | N |
| | | 10.0 | 114.7 | 33.48 ± 4.66 | N |
| | | 15.0 | 105.8 | 38.34 ± 1.70 | N |
| | 45.0 | 6.0 | 89.7 | 34.24 ± 1.31 | N |
| | | 10.0 | 94.3 | 35.18 ± 1.84 | N |
| | | 15.0 | 85.9 | 42.54 ± 3.76 | N |

It was found that only the pharmaceutical blends that contained Klucel EXAF and PEG-8000 exhibited good flow properties and produced crack-free, acceptable tablets at all compression forces. For the smallest compression force (i.e., 30 kN) crack-free tablets were only obtained for the highest pre-compression force (i.e., 15 kN). Generally, the application of pre-compression force during compression was found to minimize capping and cracking even at high compression forces.

In further experiments, tablet weights of up to 1150 mg were achieved for pharmaceutical blends containing Klucel EXAF and PEG-8000.

### Example 2

Cab-O-Sil® (i.e., colloidal silicon dioxide; source: Cabot Corporation) was selected as glidant. The pharmaceutical blends contained 80% by blend weight of sodium polystyrene sulfonate (GT160-246), 16% by blend weight PEG-8000, 3.9%, 3.8%, 3.6% or 3.4% by blend weight of Emcocel® SP15 (microcrystalline cellulose), 0%, 0.1%, 0.3% or 0.5% by blend weight Cab-O-Sil® and 0.1% by blend weight of Pruv® (i.e., sodium stearyl fumarate; source: Pewest Pharmaceutical Company). The blend ingredients were not dried before mixing. Accordingly the pharmaceutical blends contained moisture of about 5% to 6% by blend weight.

The pharmaceutical blends were compressed into capsule shaped tablets. Maximum tablet weights that were achieved are shown below:

| Cab-O-Sil ® (%) | Maximum achieved tablet weight (mg) |
|---|---|
| 0.0 | 1150 |
| 0.1 | 1336.0 |
| 0.3 | 1358.6 |
| 0.5 | 1373.8 |

The results indicate that a significantly higher tablet weight could be achieved upon incorporation of 0.1% colloidal silicon dioxide. In addition, improved blend flow characteristics were observed during compression with 0.1% colloidal silicon dioxide in the formulation. However, increasing the concentration from 0.1% to 0.5% did not further improve the flow characteristics or further increase the tablet weight significantly.

Capping or cracking on the sides of the tablets were observed for lower compression forces. A compression force of 37 kN combined with a lower pre-compression force lead to cracked tablets in some cases. A compression force of 45 kN produced crack-free tablets at all pre-compression forces.

### Example 3

Pharmaceutical blends consisting of 85.4% TOLEVAMER (i.e., copolymer of sodium polystyrene sulfonate and potassium polystyrene sulfonate), 14% Klucel® EXF (i.e., hydroxypropyl ether of cellulose; source: Hercules Inc. (Aqualon division)), 0.1% Cab-O-Sil® (i.e., colloidal silicon dioxide; source: Cabot Corporation) and 0.5% Pruv® (i.e., sodium stearyl fumarate; source: Pewest Pharmaceutical Company) were compressed into 1287 mg tablets that contained 1000 mg of anhydrous active pharmaceutical ingredient (API), that is, TOLEVAMER.

TOLEVAMER lot 1 was obtained form Hamari and lots 2 to 4 were manufactured at Haverhill using different spray drying process parameters. The physical properties of the TOLEVAMER lots, in particular, the Loss On Drying (LOD), mean particle size and volume weight mean were determined using methods known in the art. The lots differed in moisture content (i.e., Loss on Drying) and polystyrene sulfonate polymer particle sizes as shown below:

| Physical Property | Lot 1 | Lot 2 | Lot 3 | Lot 4 |
|---|---|---|---|---|
| Loss On Drying | about 5% | 5% | 8.4% | about 5% |
| Mean particle size by | 29.4 | 34.3 | 39.5 | 59.6 |
| aerosizing | | | | |
| Volume weighted mean | — | 35.6 | 42.1 | 81.6 |
| (Mastersizer) | | | | |

All lots were blended the same way. The LOD of the API was measured and then the API was blended with Klucel® EXF in a V-shell blender. The LOD of this blend was measured and a calculated amount of water was added using a micro-sprayer onto the blend under high shear mixing. The final LOD of the pharmaceutical blends was adjusted to 9%. With respect to the final moisture content of the pharmaceutical blends, it has been found that for a given pharmaceutical blend adjusted to 8%, 9% and 10% moisture content (LOD), no significant loss in difference in the compactibility results.

Tablets were prepared from the different pharmaceutical blends using three different compression forces (35 kN, 40 kN and 45 kN) and a fixed pre-compression of 15 kN to compress the blends into tablets. The tablet press speed was 40 rpm if not otherwise indicated. Tooling used for compression was an oval, shallow B-press tooling with dimensions 0.748×0.405×0.060″. This tooling was found to provide the best tablet geometry and physical characteristics. One advantage of this tooling is its shallow cup depth which was found to be better in preventing tablet cracking. An JCMCO-Healthstar 20 station instrumented B-press was used for all tablet compression purposes.

TOLEVAMER lot 4 did not compress well and weaker tablets were obtained at all compression forces at 40-rpm press speed. Tablet press speed was reduced to 20-rpm in order to evaluate if an increase in the dwell time would make an acceptable tablet.

Results for all compression runs of different lots of API are shown below:

| | Compres- | Pre- | Tablet | Compact- |
|---|---|---|---|---|
| Pharmaceutical | sion | compression | Hardness | ibility |
| blend | force (kN) | Force (kN) | (kP) | (N/kN) |
| 3 | 35 | 15 | 52.9 | 15.1 |
| | 40 | 15 | 57.5 | 14.3 |
| | 45 | 15 | 58.5 | 13.0 |
| 2 | 35 | 15 | 55.0 | 15.7 |
| | 40 | 15 | 58.4 | 14.6 |
| | 45 | 15 | 60.6 | 13.4 |
| 1 | 35 | 15 | 60.4 | 17.2 |
| | 40 | 15 | 62.6 | 15.6 |
| | 45 | 15 | 65.2 | 14.4 |
| 4 | 35 | 15 | 21.6 | 6.1 |
| | 40 | 15 | 24.9 | 6.2 |
| | 45 | 15 | 27.2 | 6.0 |
| 4 (tablet press | 35 | 15 | 30.1 | 8.0 |
| speed of 20 rpm) | 40 | 15 | 33.8 | 8.4 |
| | 45 | 15 | 33.9 | 7.5 |

The results indicate that the compression characteristics demonstrated by the TOLEVAMER lots 1, 2 and 3 were very similar. It is to be noted that different amounts of water were added to the blends containing lots 2 and 3 to achieve a final LOD of 9% and that both blends were accordingly subjected to different times of high shear mixing. However, despite this difference, no significant difference could be observed in the compactibility of above-mentioned moisture adjusted blends of two lots of API. TOLEVAMER lot 4 with an average particle size higher than the other lots did not demonstrate similar compression characteristics. The compressed tablets were weak and thick and were not acceptable for the coating process. Reduction in press speed from 40 rpm to 20 rpm in an attempt to increase the dwell time did not help in achieving comparable compactibility which indicates that as the average particle size of API increases, the compactibility of the pharmaceutical blend decreases.

These results indicate that the average particle size of polystyrene sulfonate polymer needs to be sufficiently small to allow preparation of tablets with appropriate hardness and compactibility.

Further examples of Volume weighted mean particle sizes and corresponding particle size distributions (dry basis) that have been found to be suitable in the preparation of TOLEVAMER tablets are shown below:

| | Particle Size Distribution, percentage | | | | | | | |
|---|---|---|---|---|---|---|---|---|
| Volume | of particles with partice sizes less than | | | | | | | |
| weighted mean | the size shown (based on sieve analysis) | | | | | | | |
| particle size | 300 | 180 | 150 | 106 | 75 | 45 | 38 | 20 |
| (μm) | μm | μm | μm | μm | μm | μm | μm | μm |
| 36.1 | 98.3 | 76.6 | 70.6 | 53.1 | 39.6 | 13.1 | 4.4 | 1.2 |
| 47.2 | 90.2 | 90.0 | 64.7 | 21.9 | 7.7 | 1.1 | 0.0 | 0.0 |
| 43.4 | 100.0 | 98.8 | 97.2 | 71.2 | 41.6 | 17.1 | 9.4 | 0.1 |
| 37.7 | 99.7 | 99.0 | 98.4 | 57.5 | 23.9 | 6.2 | 2.1 | 0.0 |
| 45.1 | 99.8 | 98.4 | 97.0 | 51.9 | 28.5 | 5.3 | 1.9 | 0.0 |
| 39.5 | 99.7 | 98.1 | 96.6 | 83.8 | 54.3 | 15.0 | 3.2 | 0.0 |
| 39.1 | 99.8 | 98.7 | 97.9 | 81.8 | 33.6 | 8.6 | 3.3 | 0.0 |
| 42.4 | 99.7 | 99.2 | 98.6 | 85.8 | 43.9 | 10.9 | 4.0 | 0.0 |
| 35.7 | 99.9 | 99.6 | 98.6 | 70.3 | 39.7 | 13.8 | 6.8 | 0.4 |
| 40.9 | 99.8 | 99.0 | 97.9 | 55.7 | 20.8 | 5.1 | 1.7 | 0.0 |

### Example 4

Coating materials that were evaluated included HPMC based coatings, Kollicoat® IR (i.e., an instant release coating formulation from BASF) and Opadry® II (i.e., a PVA based coating).

Coating process optimization included determining the optimum coating temperatures, airflow, pan speed and spray rate. Critical parameters for coating were the exhaust and inlet air temperatures and the time that tablets spend under these high temperature conditions. It was observed that upon subjecting the tablets to a longer coating process in order to achieve the desired weight gain, tablets tended to split or crack at the center. Accordingly, a shorter coating process and lower temperatures during coating are considered more suitable for the coating of TOLEVAMER tablets.

The HPMC based coatings (e.g., Spectrablend® consisting of a mixture of HPMC E-5, E-15 and a plasticizer) carry a low solids content (about 5%) and more water, and as a result need higher drying temperatures and generally a longer coating process. Furthermore, it has been found that HPMC based coatings lead to blistering on the surface of the tablets, which can be resolved by coating harder tablets, using slow spray rate and a high pan speed during the beginning of the coating process.

Kollicoat® IR coating has been found to yield dull finish and rough surface tablets.

For the coating of TOLEVAMER tablets coating formulations that can carry high solids content, requires low drying temperatures and dissolves sufficiently fast are desired. Opadry®-II, a PVA (polyvinyl alcohol) based coating formulation has been found to satisfy these criteria. Opadry®-II can carry a solids content of 20%, which leads for a desired weight gain of approx. 5% to a relatively short overall coating process and accordingly less time that the tablets need to spend under drying temperatures and tumbling conditions. Also, the recommended drying temperatures for Opadry®-II coating systems are lower (around 50° C. exhaust temperature). Dissolution rates with Opadry®-II were observed to be faster as compared to HPMC based coatings. Coating parameters found to work the best using Opadry®-II are as follows:

Percent solids content of coating formulation=20%
Inlet temperature=72-76° C.
Exhaust temperature=50-55° C.
Drying air=250 cfm
Atomizing air=50 psi
Spray rate=20 g/min
Pan speed 14 rpm

### Pan Load=2.5 kg

Equipment: Thomas Engineering Accela-Cota with a 19-inch pan

### Example 5

The manufacturing process for Tolevamer 1 gm tablet involved blending, wetting (moisture adjustment), moisture determination, screening, lubricating, compression, and tablet coating.

Pre-determined amounts (according to the formulations shown in the Table below) of TOLEVAMER (GT267-004), hydroxypropyl cellulose (Klucel® EXF), and colloidal silicon dioxide (Cab-O-Sil®) were dispensed and mixed in a V-shape blender for 5 minutes. The blended powder was discharged and passed through a 30 mesh stainless screen. In order to enhance the distribution of hydroxypropyl cellulose and colloidal silicon dioxide, the screened powder was reloaded back into the V-shape blender and mixed for another 5 minutes. The blended mixture was transferred into a high shear granulator for moisture adjustment. While the high shear granulator was operating with the impellor speed at 400 rpm and chopper speed at 1000 rpm, water was added into the powder blend through a micro-spray syringe. At the completion of the moisture adjustment step, the moisture content of the wetted powder mixture was examined by a halogen moisture analysis at 120° C. for 15 minutes to ensure the moisture content of the wetted blend was approximately 9%. The wetted mixture was then discharged from the granulator and passed through a co-mill equipped with a 30 mesh screen for de-lumping purpose. The co-milled mixture was lubricated with sodium stearyl furamate (Pruv®) using a V-shape blender for 5 minutes. The lubricant powder was compressed into a core tablet by using a rotary tablet press. The pre-compression force and main compression force were set at 15 kN and 35 to 45 kN, respectively. The core tablets were film coated with Opadry®-II Orange coating material using a conventional coating pan to achieve 3.5% weight gain.

| | Percentages by Tablet Weight (Dry Tablet Weight) | | | | | | | |
|---|---|---|---|---|---|---|---|---|
| Composition | A | | B | | C | | D | |
| TOLEVAMER | 76.4 | (84.0) | 82.4 | (90.6) | 84.4 | (92.8) | 86.4 | (95.0) |
| Klucel ® EXF | 14.0 | (15.4) | 8.0 | (8.8) | 6.0 | (6.6) | 4.0 | (4.4) |
| Pruv ® | 0.1 | (0.11) | 0.1 | (0.11) | 0.1 | (0.11) | 0.1 | (0.11) |
| Cab-O-Sil ® | 0.5 | (0.) | 0.5 | (0.55) | 0.5 | (0.55) | 0.5 | (0.55) |
| Water | 9 | (N/A) | 9 | (N/A) | 9 | (N/A) | 9 | (N/A) |

| | | | | |
|---|---|---|---|---|
| TOTAL | 100 | 100 | 100 | 100 |
| Tablet Weight | 1.304 g | 1.213 g | 1.185 g | 1.158 g |
| Dry Tablet Weight | 1.187 g | 1.104 g | 1.078 g | 1.054 g |
| Opadry ®-II Orange | 3.5% | 3.5% | 3.5% | 3.5% |
| coating formulation | | | | |

Composition A contained Klucel EXF with a volume weighted mean particle size in the range from 80 μm to 100 μm. Compositions B, C and D contained jet-milled (using an air-jet milling technique as known in art) Klucel EXF with volume weighted mean particle sizes in the range from 35 μm to 40 μm (measured using Mastersizer). Compositions A, B, and C resulted in acceptable tablets, whereas the tablet of composition D was not acceptable.

While this invention has been particularly shown and described with references to preferred embodiments thereof, it will be understood by those skilled in the art that various changes in form and details may be made therein without departing from the scope of the invention encompassed by the appended claims.

What is claimed is:

1. A tablet comprising at least about 70% by dry tablet weight polystyrene sulfonate polymer, binder and moisture.

2. The tablet of claim 1, wherein the binder is a hydroxypropyl ether of cellulose characterized by more than 0.4 and not more than 4.6 hydroxypropyl groups per anhydroglucose unit.

3. The tablet of claim 2 comprising about 5% to about 30% by dry tablet weight hydroxypropyl ether of cellulose.

4. The tablet of claim 3 comprising about 7% to about 13% by tablet weight moisture.

5. The tablet of claim 2, wherein about 80% to about 94% by dry tablet weight is polystyrene sulfonate polymer.

6. The tablet of claim 5 comprising about 5% to about 20% by dry tablet weight hydroxypropyl ether of cellulose.

7. The tablet of claim 6 comprising about 7% to about 12% by tablet weight moisture.

8. The tablet of claim 2, wherein about 80% to about 93.5% by dry tablet weight is polystyrene sulfonate polymer.

9. The tablet of claim 8 comprising about 6.5% to about 20% by dry tablet weight hydroxypropyl ether of cellulose.

10. The tablet of claim 9 comprising about 8% to about 12% by tablet weight moisture.

11. The tablet of claim 2, wherein about 92.8% by dry tablet weight is polystyrene sulfonate polymer.

12. The tablet of claim 11 comprising about 6.6% by dry tablet weight hydroxypropyl ether of cellulose.

13. The tablet of claim 12 comprising about 9% by tablet weight moisture.

14. The tablet of claim 1, wherein the binder is polyethylene glycol.

15. The tablet of claim 14 comprising about 5% to about 30% by dry tablet weight polyethylene glycol.

16. The tablet of claim 15 comprising about 7% to about 13% by tablet weight moisture.

17. The tablet of claim 14, wherein about 80% to about 94% by dry tablet weight is polystyrene sulfonate polymer.

18. The tablet of claim 17 comprising about 5% to about 20% by dry tablet weight polyethylene glycol.

19. The tablet of claim 18 comprising about 7% to about 12% by tablet weight moisture.

20. The tablet of claim 14, wherein about 80% to about 93.5% by dry tablet weight is polystyrene sulfonate polymer.

21. The tablet of claim 20 comprising about 6.5% to about 20% by dry tablet weight polyethylene glycol.

22. The tablet of claim 21 comprising about 8% to about 12% by tablet weight moisture.

23. The tablet of claim 1, wherein the polystyrene sulfonate polymer is sodium polystyrene sulfonate.

24. The tablet of claim 1, wherein the polystyrene sulfonate polymer is a co-polymer of sodium styrene sulfonate and potassium styrene sulfonate.

25. The tablet of claim 13, wherein the copolymer contains 30% to 80% of sodium styrene sulfonate and 70% to 20% potassium styrene sulfonate.

26. The tablet of any one of the preceding claims further comprising a glidant and a lubricant.

27. The tablet of claim 26, wherein the glidant is colloidal silicon dioxide and the lubricant is sodium stearyl fumarate.

28. The tablet of claim 27, wherein about 0.1% by dry tablet weight is colloidal silicon dioxide and about 0.5% by dry tablet weight is sodium stearyl fumarate.

29. The tablet of claim 1, wherein the tablet further comprises a coating.

30. A tablet comprising about 80% to about 94% by dry tablet weight polystyrene sulfonate polymer, about 5% to about 20% by dry tablet weight hydroxypropyl ether of cellulose characterized by more than 0.4 and not more than 4.6 hydroxypropyl groups per anhydroglucose unit, and about 7% to about 13% by tablet weight moisture.

31. The tablet of claim 30 comprising about 92.8% by dry tablet weight polystyrene sulfonate polymer, about 6.6% by dry tablet weight hydroxypropyl ether of cellulose characterized by more than 0.4 and not more than 4.6 hydroxypropyl groups per anhydroglucose unit, about 0.1% by dry tablet weight colloidal silicon dioxide, about 0.5% by dry tablet weight sodium stearyl fumarate, and about 9% by tablet weight moisture.

32. A tablet comprising about 860 mg to about 1080 mg of polystyrene sulfonate polymer, about 54 mg to about 215 mg hydroxypropyl ether of cellulose characterized by more than 0.4 and not more than 4.6 hydroxypropyl groups per anhydroglucose unit, and about 7% to about 13% by tablet weight moisture.

33. The tablet of claim 30 comprising about 1000 mg of polystyrene sulfonate polymer, about 71.1 mg hydroxypropyl ether of cellulose characterized by more than 0.4 and not more than 4.6 hydroxypropyl groups per anhydroglucose unit, about 1.19 mg colloidal silicon dioxide, about 5.93 mg sodium stearyl fumarate, and about 9% by tablet weight moisture.

34. The tablet of any one of the preceding claims, wherein the tablet contains about 1000 mg polystyrene sulfonate polymer.

35. A tablet comprising at least about 70% by dry tablet weight polystyrene sulfonate polymer, wherein the tablet has a hardness of about 30 kp to about 70 kp.

36. The tablet of claim 35 comprising about 80% to about 94% by dry tablet weight polystyrene sulfonate polymer.

37. The tablet of claim 36 wherein the hardness of the tablet is about 40 kp to about 66 kP.

38. The tablet of claim 35 comprising about 80% to about 93% by dry tablet weight polystyrene sulfonate polymer.

39. The tablet of claim 38 wherein the hardness of the tablet is about 40 kp to about 66 kP.

40. The tablet of claim 39 comprising about 92.8% by dry tablet weight polystyrene sulfonate polymer.

41. A pharmaceutical blend comprising at least about 70% by dry blend weight polystyrene sulfonate polymer and a binder.

42. The pharmaceutical blend of claim 41, wherein the binder is a hydroxypropyl ether of cellulose characterized by more than 0.4 and not more than 4.6 hydroxypropyl groups per anhydroglucose unit.

43. The pharmaceutical blend of claim 42, wherein the hydroxypropyl ether of cellulose is further characterized by a particle size distribution with a volume weighted mean particle size of about 20 μm to about 100 μm.

44. The pharmaceutical blend of claim 42, wherein the hydroxypropyl ether of cellulose is characterized by a particle size distribution with a volume weighted mean particle size of about 35 μm to about 40 μm.

45. The pharmaceutical blend of claim 41, wherein the polystyrene sulfonate polymer is characterized by a particle size distribution with a mean particle size of about 15 μm to about 70 μm and a volume weighted mean particle size of about 30 μm to about 90 μm.

46. The pharmaceutical blend of claim 45, wherein the polystyrene sulfonate polymer is characterized by a particle size distribution with a mean particle size of about 25 μm to about 50 μm and a volume weighted mean particle size of about 40 μm to about 60 μm.

47. The pharmaceutical blend of claim 42, wherein the polystyrene sulfonate polymer is characterized by a particle size distribution with a mean particle size of about 15 μm to about 70 μm and a volume weighted mean particle size of about 30 μm to about 90 μm and the hydroxypropyl ether of cellulose is further characterized by a particle size distribution with a volume weighted mean particle size of about 20 μm to about 100 μm.

48. The pharmaceutical blend of claim 47, wherein the polystyrene sulfonate polymer is characterized by a particle size distribution with a mean particle size of about 25 μm to about 50 μm and a volume weighted mean particle size of about 40 μm to about 60 μm and the hydroxypropyl ether of cellulose is further characterized by a particle size distribution with a volume weighted mean particle size of about 35 μm to about 40 μm.

49. The pharmaceutical blend of claim 42 comprising about 5% to about 30% by dry blend weight hydroxypropyl ether of cellulose.

50. The pharmaceutical blend of claim 49 further comprising about 7% to about 13% by blend weight moisture.

51. The pharmaceutical blend of claim 42, wherein about 80% to about 94% by dry blend weight is polystyrene sulfonate polymer.

52. The pharmaceutical blend of claim 51 comprising about 5% to about 20% by dry blend weight hydroxypropyl ether of cellulose.

53. The pharmaceutical blend of claim 52 further comprising about 7% to about 12% by blend weight moisture

54. The pharmaceutical blend of claim 42, wherein about 80% to about 93.5% by dry blend weight is polystyrene sulfonate polymer.

55. The pharmaceutical blend of claim 54 comprising about 6.5% to about 20% by dry blend weight hydroxypropyl ether of cellulose.

56. The pharmaceutical blend of claim 55 further comprising about 8% to about 12% by blend weight moisture

57. The pharmaceutical blend of claim 42, wherein about 92.8% by dry blend weight is polystyrene sulfonate polymer.

58. The pharmaceutical blend of claim 57 comprising about 6.6% by dry blend weight hydroxypropyl ether of cellulose.

59. The pharmaceutical blend of claim 58 further comprising about 9% by blend weight moisture.

60. The pharmaceutical blend of claim 41, wherein the binder is polyethylene glycol.

61. The pharmaceutical blend of claim 60 comprising about 8% to 30% by dry blend weight polyethylene glycol.

62. The pharmaceutical blend of claim 61 further comprising about 7% to about 13% by blend weight moisture.

63. The pharmaceutical blend of claim 60, wherein about 80% to about 90% by dry blend weight is polystyrene sulfonate polymer.

64. The pharmaceutical blend of claim 63 comprising about 10% to about 20% (by dry blend weight) polyethylene glycol.

65. The pharmaceutical blend of claim 64 further comprising about 8% to about 12% by blend weight moisture.

66. The pharmaceutical blend of claim 41, wherein the polystyrene sulfonate polymer is sodium polystyrene sulfonate.

67. The pharmaceutical blend of claim 41, wherein the polystyrene sulfonate polymer is a co-polymer of sodium styrene sulfonate and potassium styrene sulfonate.

68. The pharmaceutical blend of claim 67, wherein the copolymer contains 30% to 80% of sodium styrene sulfonate and 70% to 20% potassium styrene sulfonate.

69. The pharmaceutical blend of claim 41, wherein the blend has a compactibility of about 9 N/kN to about 20 N/kN.

70. A method of preparing a tablet containing polystyrene sulfonate polymer comprising the step of compressing the pharmaceutical blend of any one of claims 36-57 with a compression force of about 25 kN to about 60 kN to form a tablet.

71. The method of claim 70, wherein the pharmaceutical blend is pre-compressed with a pre-compression force of about 5 kN to about 30 kN.

72. The method of claim 70, wherein the compression force is about 35 kN to about 50 kN.

73. The method of claim 70, wherein the pharmaceutical blend is pre-compressed with a pre-compression force of about 10 kN to about 20 kN.

74. A method of treating a bacterial infection in a subject, the bacterial infection being characterized by release of a pathogenic toxin, comprising the step of administering to the subject a therapeutically effective number of tablets of any one of claims 1-40.

75. The method of claim 74 wherein the pathogenic toxin is released from a bacterium selected from the group consisting of Streptococcus spp.; Salmonella spp.; Campylobacter spp.; Escherichia spp.; Clostridia spp.; Vibrio spp.; Staphylococcus spp.; Shigella spp.; Pseudomonas spp.; Bordatella spp.; Listeria spp.; Yersinia spp.; Legionella spp.; Bacillus spp.; Helicobacter spp.; Corynebacteria spp.; Actinobacillus spp.; Aeromonas spp.; Bacteroides spp. and Pasteurella spp.

76. The method of claim 75 wherein the pathogenic toxin is released from a bacterium selected from the group consisting of Streptococcus pneumoniae, Streptococcus pyogenes, Salmonella enteritidis, Campylobacter jejuni, Escherichia coli, Clostridium botulinum, Staphylococcus aureus, Shigella dysenteriae, Pseudomonas aeruginosa, Bordatella pertussis, Listeria monocytogenes, Yersinia enterocolitica, Legionella pneumophilia and Bacillus anthracis.

77. The method of claim 74 wherein the pathogenic toxin is released from Clostridium difficile.

78. A method of treating antibiotic-associated diarrhea in a subject comprising administering to the subject a therapeutically effective number of tablets of any one of claims 1-40.

79. A method of treating Clostridium difficile associated diarrhea in a subject comprising administering to the subject a therapeutically effective number of tablets of any one of claims 1-40.
